(19) United States
(12) Patent Application Publication
(10) Pub. No.: US 2005/0148960 A1
(43) Pub. Date: Jul. 7, 2005

(54) Absorbent pad with three-dimensional end containment

(75) Inventor: Cindy Lou Price, Appleton, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 10/741,755

(22) Filed: Dec. 18, 2003

(51) Int. Cl. A61F 13/20

(52) U.S. Cl. 604/358

(57) ABSTRACT

An absorbent pad is configured for attachment in an undergarment and used to absorb bodily exudates. The pad has a backsheet layer, topsheet layer, an absorbent assembly, and a three-dimensional capture structure. The three-dimensional capture structure is located in the rear portion of the pad on the body-facing surface and is adapted to conform to a wearer's gluteal groove when the rear portion of the absorbent pad is positioned in an undergarment adjacent a wearer's perianal region.

### BACKGROUND

Absorbent pads for menstruation or urinary incontinence are intended to absorb and retain bodily exudates from a wearer. Such pads typically have an absorbent structure disposed between a fluid (or liquid) permeable topsheet and a fluid (or liquid) impermeable backsheet. The pads are generally adapted to fit between the legs of the wearer and are placed near the vaginal and/or urethral openings to retain body fluids. However, despite the proximity to the body of the wearer, leakage at the rear of the pad has been a problem in use. On occasions, not all the fluid is absorbed rapidly enough, rather, a portion of the fluid can run along the surface of the pad and leak out the rear of the pad. On other occasions, fluid discharge may stream across the surface of the wearer's body, with some leaking out the rear of the pad. In either situation, rear leakage is worsened when the wearer is in a reclined position or is prone.

Various efforts have been undertaken to address leakage in general, including a variety of pockets, dams, channels, and recesses. Other efforts have included deformation elements that rely primarily upon lateral compressive forces to cause the entire absorbent article to fold and bow upward towards the wearer. Despite these efforts, rear leakage is still a frequent means of product failure. Particularly troublesome is leakage near the gluteal groove. The gluteal groove forms a natural channel that provides a route for fluids to follow and factors into the problem of rear leakage with absorbent pads. Thus, there is a need for an absorbent pad with improved protection against rear leakage and specifically improved protection along the gluteal groove. There is also a need for an absorbent pad that provides additional protection against rear leakage when the wearer is prone or reclined.

### SUMMARY

In response to the difficulties and problems discussed above, a new absorbent pad has been discovered. The absorbent pad is configured for attachment in an undergarment and has a rear portion, front portion, garment-facing surface, and body-facing surface. The pad has a backsheet layer, a liquid permeable topsheet layer secured to and in facing relation with the backsheet layer, an absorbent assembly disposed between the backsheet layer and the topsheet layer, and a three-dimensional capture structure disposed in the rear portion of the pad on the body facing surface. The three-dimensional capture structure is adapted to conform to a wearer's gluteal groove when the rear portion of the absorbent pad is positioned in an undergarment adjacent a wearer's perianal region.

The absorbent pad, in some embodiments, has elasticized side retention members disposed outboard of the absorbent assembly. The elasticized side retention members apply tension to the three-dimensional capture structure and facilitate opening the three-dimensional capture structure.

The three-dimensional capture structure, in various embodiments, also includes a spacer. In various embodiments, the three-dimensional capture structure is a unitary structure constructed of the backsheet layer and the topsheet layer. In other embodiments, the three-dimensional capture structure is a non-unitary structure constructed of one or more additional materials attached to the absorbent pad. The three-dimensional capture structure also has one or more capture elastics in some embodiments.

In some embodiments, the liquid permeable topsheet layer is disposed primarily on the body-facing surface and the backsheet layer is disposed partially on the body-facing surface and partially on the garment-facing surface. As such, the portion of the backsheet layer disposed on the garment-facing surface has an area A1 and the portion of the backsheet layer disposed on the body-facing surface has an area A2; and the ratio A1/A2 is less than about 7 to 1. In some embodiments, the ratio A1/A2 is less than about 6 to 1, less than about 5 to 1, or less than about 4 to 1.

In some embodiments, the three-dimensional capture structure has a width and a height and the ratio of the width to the height is less than about 4 to 1, less than about 3 to 1, less than about 2 to 1, or less than about 1 to 1.

In various embodiments, the absorbent pad is configured for attachment in an undergarment and the pad has a rear portion, front portion, garment-facing surface, and body-facing surface. The pad has a backsheet layer, a liquid permeable topsheet layer secured to and in facing relation with the backsheet layer; an absorbent assembly disposed between the backsheet layer and the topsheet layer, a garment attachment mechanism disposed on the backsheet layer remote from the absorbent assembly, elasticized side retention members disposed outboard of the absorbent assembly, and a three-dimensional capture structure disposed in the rear portion of the pad and on the body-facing surface. The three-dimensional capture structure is constructed of the backsheet layer and the topsheet layer folded and joined to form a generally pyramidal shape and adapted to conform to a wearer's gluteal groove when the rear portion of the absorbent pad is positioned in an undergarment adjacent a wearer's perianal region. In various embodiments configured as such, the three-dimensional capture structure defines a volume. In various embodiments, the three-dimensional capture structure includes one or more capture elastics adapted to maintain the volume when the rear portion of the absorbent pad is positioned in a wearer's perianal region. Additionally, the three-dimensional capture structure, of some of these embodiments, includes a spacer. In various of these embodiments, the backsheet layer is present in at least a portion of the side retention members.

Further in response to the difficulties and problems discussed above, a new method of providing a system of reducing leakage from the rear portion of an absorbent pad has been discovered. The method provides an absorbent pad configured for attachment in an undergarment. The absorbent pad has a rear portion, front portion, garment facing surface, and body facing surface. The pad has a backsheet layer, a liquid permeable topsheet layer secured to and in facing relation with the backsheet layer, an absorbent assembly disposed between the backsheet layer and the topsheet layer, and a three-dimensional capture structure disposed in the rear portion of the pad on the body facing surface. The three-dimensional capture structure is adapted to conform to a wearer's gluteal groove when the rear portion of the absorbent pad is positioned in an undergarment adjacent a wearer's perianal region. The method further provides instructions directing a wearer to attach the absorbent pad in an undergarment and position the absorbent pad such that the three-dimensional capture structure is adjacent the wearer's perianal region. The method enables the wearer to position the absorbent pad in the undergarment by following the instructions provided so as to reduce rear leakage.

### FIGURES

FIG. 1 representatively illustrates a perspective view of one embodiment of an exemplary absorbent pad.

FIG. 2 representatively illustrates a longitudinal section view taken along line 2-2 of FIG. 1.

FIG. 3 representatively illustrates a transverse section view taken along line 3-3 of FIG. 1.

FIG. 3A representatively illustrates a transverse section view taken along line 3A-3A of FIG. 1.

FIG. 4 representatively illustrates a top plan view of one embodiment of an exemplary absorbent pad taken from the body-facing side of the pad and in a laid flat condition, with portions broken away for purposes of illustration.

FIG. 5 representatively illustrates a bottom plan view of one embodiment of an exemplary absorbent pad taken from the garment-facing side of the pad and in a laid flat condition, with portions broken away for purposes of illustration.

FIG. 6 representatively illustrates a top plan view of an interim step in the formation of one embodiment of an exemplary absorbent pad taken from the body-facing side of the pad and in a laid flat condition, with portions broken away for purposes of illustration.

FIG. 7 representatively illustrates a top plan view of an interim step in the formation of one embodiment of an exemplary absorbent pad taken from the body-facing side of the pad and in a laid flat condition, with portions broken away for purposes of illustration.

FIG. 8 representatively illustrates a rear view of one embodiment of an exemplary absorbent pad positioned in the perianal region of a wearer.

FIG. 8A representatively illustrates a top plan view of the rear portion of one embodiment of an exemplary absorbent pad.

FIG. 8B representatively illustrates a top plan view of the rear portion of the embodiment of FIG. 8A with force being applied.

FIG. 9 representatively illustrates a rear view of an absorbent pad without a three-dimensional capture structure positioned in the perianal region of a wearer.

FIG. 10 representatively illustrates a top plan view of an interim step in the formation of one embodiment of an exemplary absorbent pad taken from the body-facing side of the pad and in a laid flat condition.

FIG. 11 representatively illustrates a top plan view of an interim step in the formation of one embodiment of an exemplary absorbent pad taken from the body-facing side of the pad and in a laid flat condition.

FIG. 12 representatively illustrates a top plan view of one embodiment of an exemplary absorbent pad taken from the body-facing side of the pad and in a laid flat condition.

FIGS. 13 A-J representatively illustrate various embodiments of exemplary absorbent pads to clarify the width, height, and area measurements disclosed herein.

FIG. 14 representatively illustrates a top plan view of the rear portion of one embodiment of an exemplary absorbent pad with portions broken away for purposes of illustration.

FIG. 15 representatively illustrates a top plan view of the rear portion of one embodiment of an exemplary absorbent pad.

FIG. 15A representatively illustrates a transverse section view taken along line 15A-15A of FIG. 15.

FIG. 16 representatively illustrates a top plan view of an exemplary absorbent pad taken from the body-facing side of the pad and in a laid flat condition.

FIG. 17 representatively illustrates a transverse section view taken along line 17-17 of FIG. 16.

FIG. 18 representatively illustrates a perspective view of the rear portion of one embodiment of an exemplary absorbent pad.

FIG. 18A representatively illustrates a transverse section view taken along line 18A-18A of FIG. 18.

FIG. 18B representatively illustrates an alternative transverse section view taken along line 18A-18A of FIG. 18.

FIG. 19 representatively illustrates a perspective view of the rear portion of an exemplary absorbent pad.

FIG. 19A representatively illustrates a transverse section view taken along line 19A-19A of FIG. 19.

FIG. 19B representatively illustrates an enlarged transverse section view of the spacer of an exemplary absorbent pad.

### DETAILED DESCRIPTION

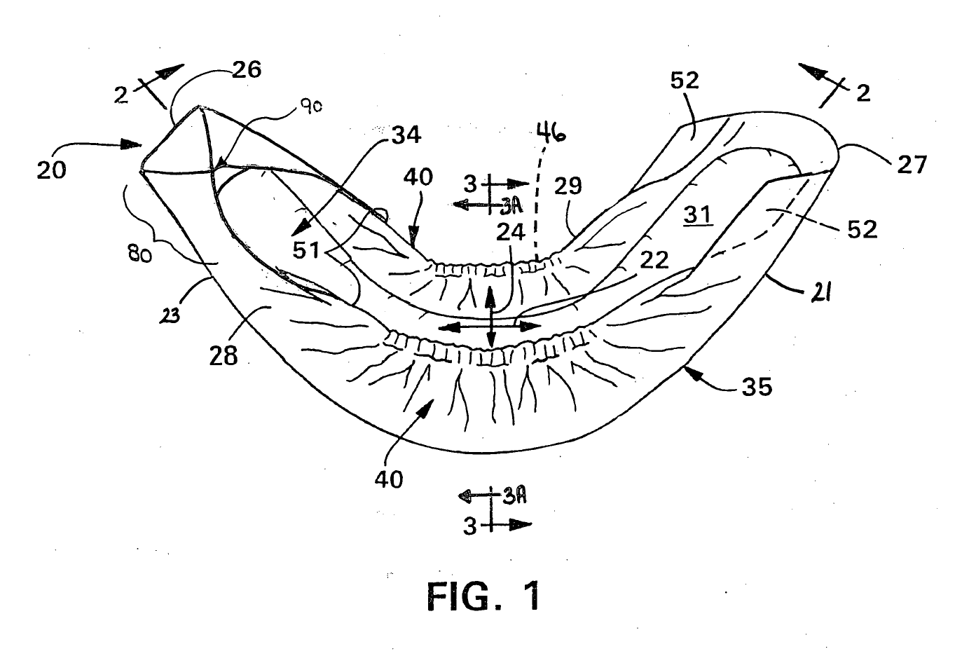

With reference generally to FIGS. 1-5, an absorbent article formed according to the present invention is shown for purposes of illustration as a disposable absorbent pad 20 for adult incontinence. As used herein, the term “absorbent pad” means an article generally sized and shaped to fit in the crotch region of a wearer and generally configured to be worn with an undergarment, such as underwear or panty hose. An absorbent pad is used to absorb bodily exudates that come into contact with the pad. An absorbent pad should be distinguished from a full-length absorbent article such as a baby diaper or adult incontinence briefs or absorbent undergarments, which are generally not configured for use with underwear. Embodiments of the invention may be adapted specifically for absorption of menses, blood, or other body excrement. For example, the invention may be embodied in an absorbent feminine hygiene article such as a sanitary napkin or urinary incontinence article.

Referring specifically to FIG. 1, the illustrated absorbent pad 20 defines a longitudinal axis or centerline represented by arrow 22, which generally corresponds to the greatest planar dimension of the pad. A perpendicular transverse axis or centerline is represented by arrow 24. The absorbent pad 20 has a front portion 21 and a rear portion 23. The absorbent pad 20 also has opposite longitudinal end edges 26 and 27 and opposite side edges 28 and 29. The longitudinal end edge 26 is in the rear portion 23 and the longitudinal end edge 27 is in the front portion 21. The longitudinal side edges 28 and 29 extend between the longitudinal end edges 26 and 27. The longitudinal end edges 26 and 27 and the longitudinal side edges 28 and 29 may be straight, curvilinear or irregularly shaped. Suitable absorbent pads have a length dimension measured between the longitudinal end edges 26 and 27 of from about 10 cm to about 65 cm, from about 20 cm to about 50 cm, or from about 30 cm to about 40 cm. Suitable absorbent pads have a width dimension measured between the longitudinal side edges 28 and 29 of from about 3 cm to about 23 cm, from about 5 cm to about 17 cm, or from about 7 cm to about 13 cm.

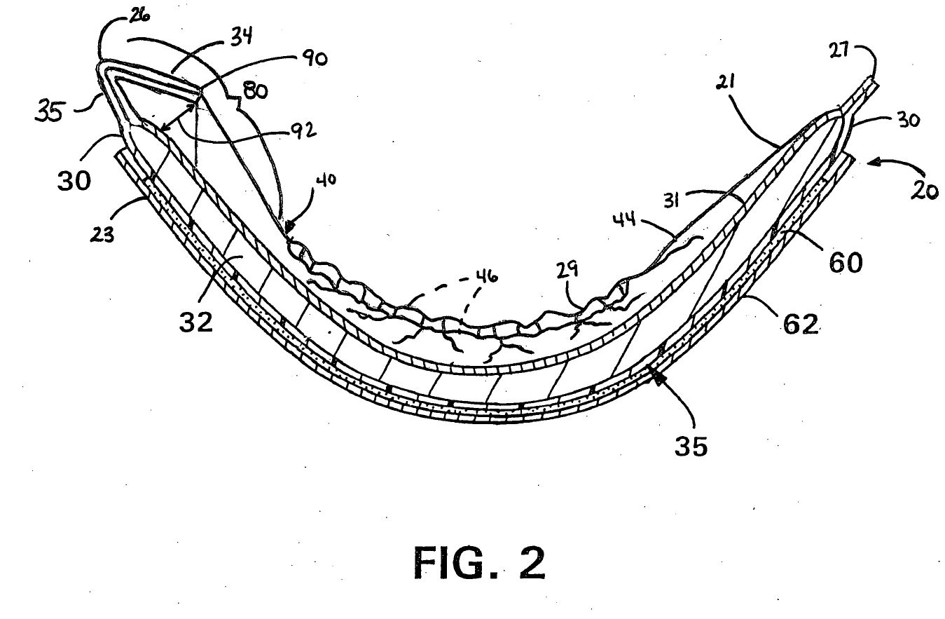

FIG. 2 representatively illustrates a longitudinal cross section of the absorbent pad 20 of FIG. 1 taken along the line 2-2. The absorbent pad 20 includes a substantially fluid impermeable backsheet layer 30, a fluid (or liquid) permeable topsheet layer 31 superposed on the backsheet layer 30, and an absorbent assembly 32 sandwiched between the backsheet layer 30 and the topsheet layer 31. The backsheet and topsheet layers 30 and 31 are desirably longer and wider than the absorbent assembly 32. The topsheet layer 31 is designed to be generally positioned toward the wearer which is referred to as the body-facing surface 34. Conversely, the backsheet layer 30 is designed to be generally positioned toward the undergarment of the wearer which is referred to as the garment-facing surface 35. However, in various configurations, a portion of the backsheet layer 30 may be positioned towards the body-facing surface 34, or a portion of the topsheet layer 31 may be positioned towards the garment-facing surface 35, or both.

The backsheet layer 30 may be made of a material that is formed or treated to be fluid impermeable. Alternatively, the backsheet layer 30 may be made of a fluid permeable material and other suitable means (not shown), such as a fluid impermeable layer associated with the absorbent assembly 32, may be provided to impede fluid movement away from the absorbent assembly 32. The backsheet layer 30 may be a single layer of material or a laminate of two or more separate layers of material. Suitable backsheet layer materials include films, wovens, nonwovens, laminates of films, wovens, and/or nonwovens, rubber sheets, or the like, or combinations thereof. For example, the backsheet layer 30 may be a thin, substantially fluid impermeable web or sheet of plastic film such as polyethylene, polypropylene, or similar material. One suitable material for the backsheet layer 30 is a 0.028 millimeter (mm) thick polyethylene film with a systematic matte embossed pattern and that has been corona treated on both sides. The terms “fluid impermeable” or “liquid impermeable” are used herein to describe a layer or laminate having a hydrohead of at least about 30 cm. Hydrohead is determined according to Federal Test Standard 191A, Method 5514 using a Testest FX-3000 Hydrostatic Head Tester available from Marlo Industries, Inc., PO Box 1071, Concord, N.C. The term “nonwoven web” means a web of fibers or filaments that is formed by means other than knitting or weaving and that contains bonds between some or all of the fibers or filaments; such bonds may be formed, for example, by thermal, adhesive, or mechanical means such as entanglement. A nonwoven web has a structure of individual fibers or threads that are interlaid, but not in an identifiable repeating manner.

In some embodiments, the backsheet layer 30 may be made of elastomeric materials. The backsheet layer 30 may, for example, be composed of a single layer, multiple layers, laminates, spunbond fabrics, films, meltblown fabrics, elastic netting, microporous web, bonded carded webs or foams made of elastomeric or polymeric materials. Elastomeric nonwoven laminate webs may include a nonwoven material joined to one or more gatherable nonwoven webs, films, or foams. Stretch Bonded Laminates (SBL) and Neck Bonded Laminates (NBL) are examples of elastomeric composites. Examples of suitable materials are Spunbond-Meltblown fabrics, Spunbond-Meltblown-Spunbond fabrics, Spunbond fabrics, or laminates of such fabrics with films, foams, or other nonwoven webs. Elastomeric materials may include cast or blown films, foams, or meltblown fabrics composed of polyethylene, polypropylene, or polyolefin copolymers, as well as combinations thereof. The elastomeric materials may include PEBAX elastomer (available from AtoChem located in Philadelphia, Pa.), HYTREL elastomeric polyester (available from E. I. DuPont de Nemous located in Wilmington, Del.), KRATON elastomer (available from Shell Chemical Company located in Houston, Tex.), or strands of LYCRA elastomer (available from E. I. DuPont de Nemous located in Wilmington, Del.), or the like, as well as combinations thereof. The backsheet layer 30 may include materials that have elastomer properties through a mechanical process, printing process, heating process, or chemical treatment. For example such materials may be apertured, creped, neck-stretched, heat activated, embossed, and micro- strained; and may be in the form of films, webs, and laminates.

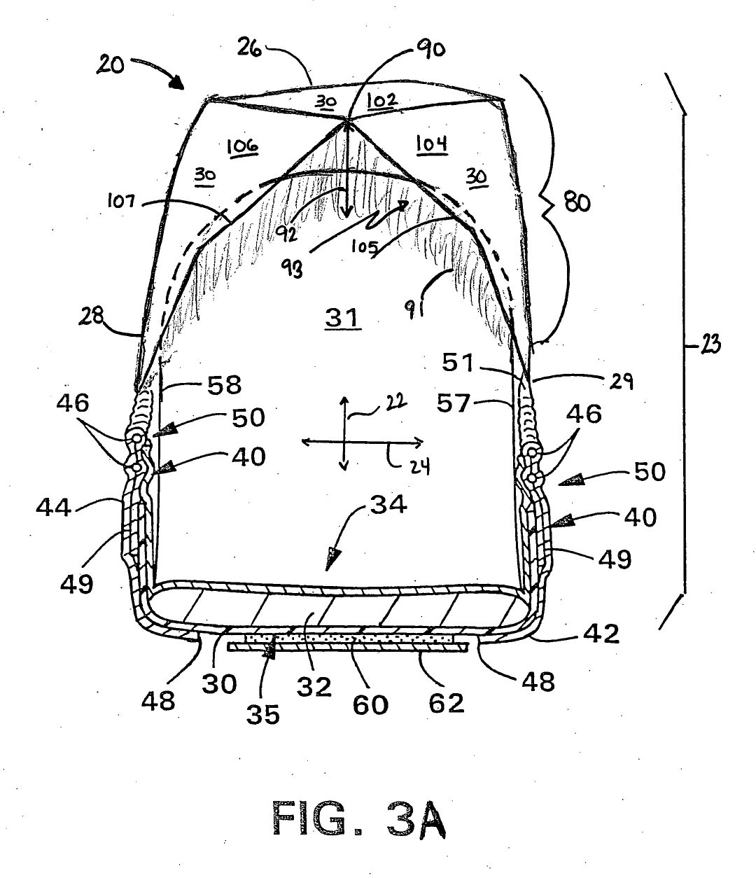

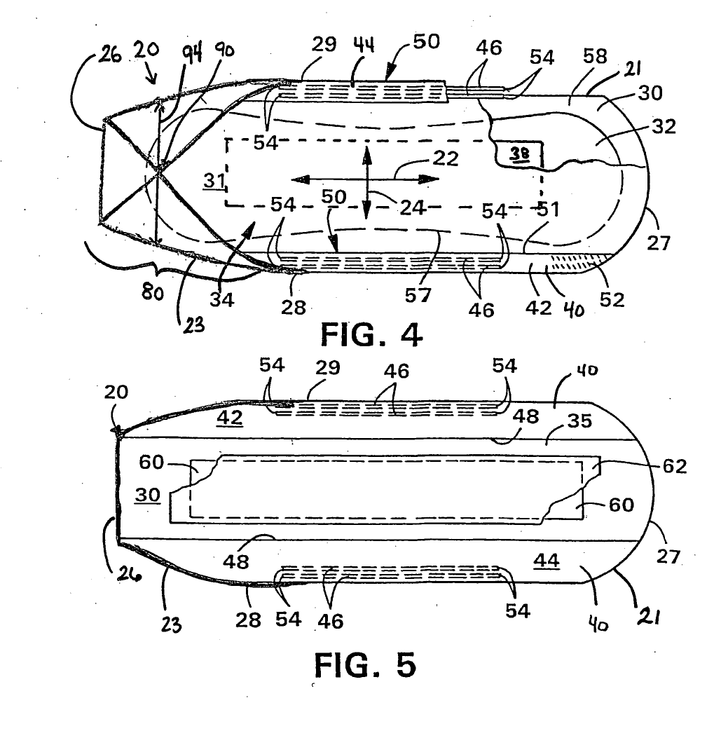

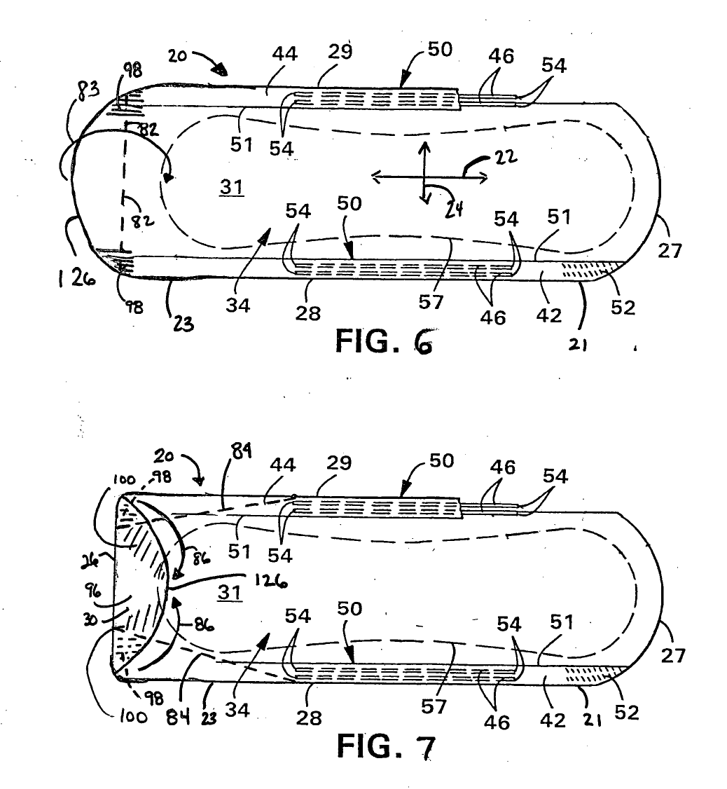

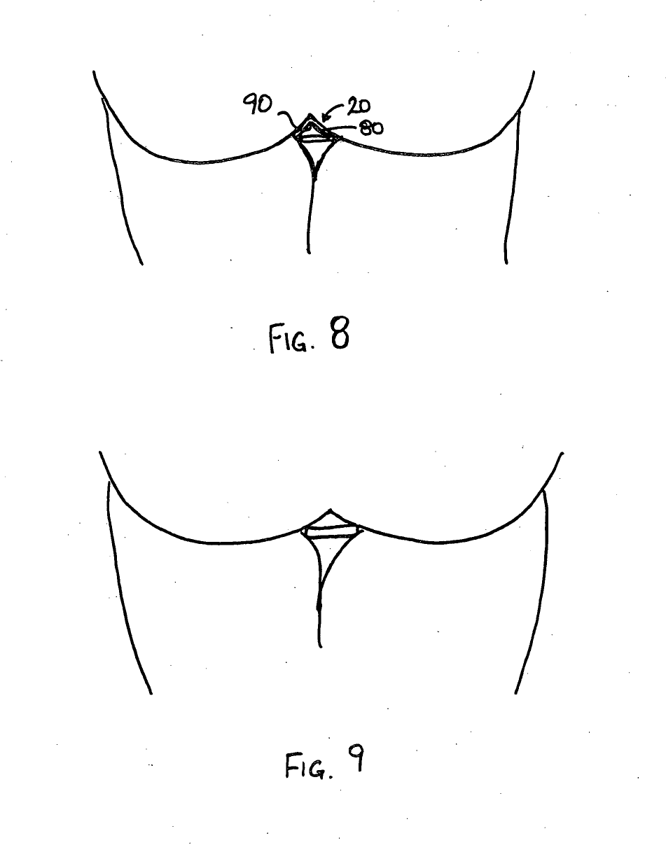

The absorbent assembly 32 is made of materials adapted to absorb and retain urine, menses, blood, or other body excrement. The absorbent assembly 32 may contain various natural or synthetic absorbent materials, such as cellulose fibers, surfactant treated meltblown fibers, wood pulp fibers, regenerated cellulose or cotton fibers, a blend of pulp and other fiber, or the like, or combinations thereof. One such material is a coform material which is composed of a mixture of cellulosic fibers and synthetic polymer fibers. The absorbent assembly 32 may also include compounds to increase its absorbency, such as 0-95 weight percent of organic or inorganic high-absorbency materials, commonly referred to as “superabsorbents”, which are typically capable of absorbing at least about 15 and desirably more than 25 times their weight in water. Suitable high-absorbency materials are described in U.S. Pat. No. 4,699,823 issued Oct. 13, 1987, to Kellenberger et al. and U.S. Pat. No. 5,147,343 issued Sep. 15, 1992, to Kellenberger, which are incorporated herein by reference. High-absorbency materials are available from various commercial vendors, such as Dow Chemical Company, Hoechst Celanese Corporation, and Allied Colloids, Inc. The absorbent assembly 32 may also include tissue layers (not shown) or an acquisition or distribution layer 38, as illustrated in FIG. 4, to help maintain the integrity of fibrous absorbents or transport fluids. Other suitable combinations for absorbent assembly 32 for the absorbent pad 20 are described in U.S. Pat. No. 6,315,765 issued Nov. 13, 2001 to Datta et al., which is incorporated herein by reference in its entirety.

The topsheet layer 31 is formed of a fluid permeable material so that fluid waste, and possibly semi-solid waste as well, can pass through and be absorbed by the absorbent assembly 32. Suitable topsheet layers 31 may be made of a nonwoven web, a spunbond, meltblown or bonded-carded web composed of synthetic polymer filaments or fibers, such as polypropylene, polyethylene, polyesters or the like, a perforated film, or a web of natural polymer filaments or fibers such as rayon or cotton or combinations thereof. In addition, the topsheet layer 31 may be treated with a surfactant to aid in fluid transfer. In one particular embodiment, the topsheet layer 31 is made of a nonwoven, spunbond web of side-by-side bicomponent filaments with 50 percent polyethylene and 50 percent polypropylene having a basis weight of about 20 grams per square meter (gsm). The fabric is surface treated with a surfactant commercially available from Union Carbide Chemicals and Plastics Company, Inc., of Danbury, Conn., U.S.A. under the trade designation TRITON X-102. As used herein, the term “fabric” refers to all of the woven, knitted and nonwoven fibrous webs.

In particular aspects, the topsheet layer 31 may be made of elastomeric materials such as elastic strands, LYCRA elastics, elastic films, cast or blown; nonwoven elastic webs, meltblown or spunbond elastomeric fibrous webs, as well as combinations thereof. Examples of elastomeric materials include KRATON elastomers, HYTREL elastomers, ESTANE elastomeric polyurethanes (available from B.F. Goodrich and Company located in Cleveland, Ohio), or PEBAX elastomers. The topsheet 31 layer may include blends or laminates of fibers, scrim, webs, and films with perforations, apertures, creping, heat activation, embossing, micro-straining, chemical treatment, or the like, as well as combinations thereof.

Referring to FIG. 1, the absorbent pad 20 includes a three-dimensional capture structure 80 disposed along and integral with the end edge 26 and disposed generally on the body-facing surface 34. The absorbent pad 20 may also include a pair of side retention members 40 disposed along and forming the side edges 28 and 29 of the pad. The side retention members 40 are disposed transversely outward of the absorbent assembly 32 and may be formed partially or completely by portions of the backsheet layer 30 and/or the topsheet layer 31. Alternatively, the side retention members 40 can be formed by portions of two side panel members 42 and 44, as illustrated in FIGS. 4 and 5. The side retention members 40 can also include elastic members 46 adapted to gather portions of the longitudinal side edges 28 and 29, as illustrated in FIGS. I and 2. In such embodiments, the side retention members 40 are considered to be “elasticized.”

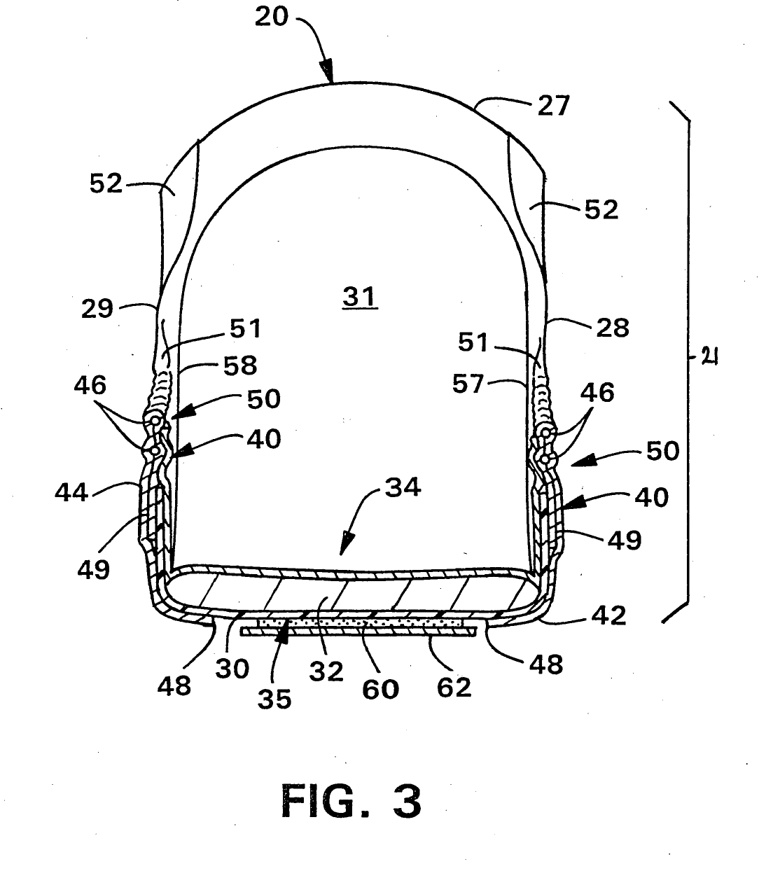

Referring now to FIG. 4, the separate side panel members 42 and 44 may be bonded to the backsheet layer 30 transversely outward from the longitudinal centerline 22. Each side panel member 42 and 44 includes a proximal edge 48 and an opposite distal edge 49, as illustrated in FIGS. 3 and 3A. In the illustrated embodiment, the proximal edges 48 are disposed on the garment-facing surface 35 of the absorbent pad 20, and the distal edges 49 are disposed in the side retention members 40. Adjacent the distal edges 49, the side panel members 42 and 44 desirably include a folded region 50 to encase the elastic members 46. The folded region 50 is thus raised above the plane of the absorbent assembly 32 and defines a free edge 51 of each side retention member 40. The side panel members 42 and 44 are desirably formed of a soft, gatherable material such as a spunbond nonwoven or the like. In one particular embodiment, the side panel members 42 and 44 are made of a high loft fuzzy nonwoven spunbond made of side-by-side bicomponent filaments of 50 percent polyethylene and 50 percent polypropylene.

As best illustrated in FIGS. 4 and 5, the side retention members 40 extend the full length of the absorbent pad 20. The side retention members 40 may be folded and bonded to itself near the longitudinal end edge 27 forming bonded regions 52 in order to reduce the quantity of free material in the corners. The bonded regions 52 of the side retention members 40 that are bonded to themselves are illustrated with cross hatched lines in FIG. 4, and the resulting inversion of the side retention members is shown in FIG. 1. Alternatively, the longitudinal ends of the side retention members 40 may be folded over and bonded onto the body-facing side of the absorbent assembly 32, or may not be folded and bonded in place at all (not shown).

The length and width of the fluid impermeable backsheet layer 30 may be selected so that the backsheet layer 30 is present in at least a portion of the side retention members 40, or in at least a portion of the three-dimensional capture structure 80, or in at least a portion of both. The backsheet layer 30 may be completely covered by either the topsheet layer 31 or the side panel members 42 and 44. Alternatively, the side retention members 40, or the three-dimensional capture structure 80, or both may be formed at least partially of the topsheet layer 31, the side panel members 42 and 44, the backsheet layer 30, or combinations thereof.

In embodiments including elasticized side retention members 40, the elastic members 46 are desirably operable over less than the full length of the absorbent pad 20. Each elastic member 46 has opposite active ends 54 (FIGS. 4 and 5) that are longitudinally spaced from the longitudinal end edges of the absorbent pad 20. As used herein, the term “active ends” refers to the terminal points of the elastic members 46 that are attached to other materials of the side retention member 40 and between which the elastic member 46 is effective to gather together the side retention member 40.

The elastic members 46 are longitudinally orientated in each side retention member 40 and operatively joined to the side retention member 40. The elastic members 46 may be bonded to the side retention member 40 in a stretched condition, bonded in a relaxed state to a gathered portion of the side retention member 40, or may have elastic properties activated after being bonded to the side retention member 40. “Operatively joined,” with reference to the attachment of an elastic member to another element, means that the elastic member when attached to or connected to the element, or treated with heat or chemicals, by stretching, or the like, gives the element elastic properties; and with reference to the attachment of a non-elastic member to another element, means that the member and element can be attached in any suitable manner that permits or allows them to perform the intended or described function of the joining. The joining, attaching, connecting or the like can be either directly, such as joining either member directly to an element, or can be indirectly by means of another member disposed between the first member and the first element.

The elastic members 46 may be formed of a dry-spun coalesced multifilament elastomeric thread sold under the tradename LYCRA and available from E.I. Du Pont de Nemours and Company. Alternately, the elastic members 46 may be formed of other typical elastics utilized in making incontinence products, such as a thin ribbon of natural rubber, wet-spun spandex materials, a stretch bonded laminate material comprising a prestretched elastic meltblown inner layer sandwiched between and bonded to a pair of spunbond polypropylene nonwoven webs, or the like. Elasticity could also be imparted to the absorbent article by extruding a hot melt elastomeric adhesive on the side retention members 40.

To improve performance of the side retention members 40 in particular and the absorbent pad 20 overall, the elastic members 46 of each side retention member 40 are desirably positioned at least partly out of the plane of the absorbent assembly 32 when the absorbent pad 20 is in a generally flat position, as the absorbent pad 20 would be when worn by an adult. In general, an elastic member 46 is considered to be at least partly out of the plane of the absorbent assembly 32 when, at some point at or between the active ends 54, a portion of the elastic member 46 is disposed above the top surface of the absorbent assembly 32. The configuration of the side retention members 40 is such that the elastic members 46 rise at least partially out of the plane of the absorbent assembly 32, allowing the side retention members 40 and the elastic members 46 to fit against or closer to the body of the wearer between the genitalia and the legs thereby providing a gasketing contact between the side retention members 40 and the wearer's body. The gasketing contact is provided toward the abdomen and the buttocks along nearly the entire length of the absorbent pad 20.

In addition, the configuration of the side retention members 40 of the present invention provides a bucket shaped structure as illustrated in FIG. 1. The side retention members 40 serve to stop or impede fluid migration away from the absorbent assembly 32 during fluid insults before the fluid has been absorbed into the absorbent assembly 32. During this time, the fluid may flow or migrate to the edges or ends of the absorbent article. The side retention members 40 function as a dam or barrier, retaining the fluid at the edges in proximity of the absorbent assembly 32 until the fluid can be absorbed. Because the elastic members 46 and the side retention members 40 extend above the plane of the absorbent assembly 32, a bucket shaped structure is formed in which the fluid can be retained until it is absorbed by the absorbent pad 20.

The absorbent pad 20 also includes a three-dimensional capture structure 80 in the rear portion 23 and disposed along and integral with the longitudinal end edge 26 of the absorbent pad 20 as representatively illustrated in FIGS. 1, 2, 3A, and 4. The three-dimensional capture structure 80 is generally disposed on the body-facing surface 34 of the absorbent pad 20 and extends at least partially in the z-direction. As used herein, the “z-direction” refers to the direction perpendicular to the plane created by the longitudinal axis 22 and the transverse axis 24. As used herein, the term “three-dimensional” refers to a structure having at least one length dimension, width dimension, or height dimension extending generally along each of the longitudinal axis 22, the transverse axis 24, and the z-direction. As used herein, the term “capture structure” means an assembly or device having walls, panels, sides, or similar structure configured to define a volume and to define an entrance to the volume disposed in such a manner so as to capture and retain fluid within the volume after the fluid has passed through the entrance. The three-dimensional capture structure 80 is adapted to reduce leakage at the rear of the absorbent pad 20 by retaining urine, menses, or other body exudates that may migrate along the topsheet layer 31 of the absorbent pad 20 or along the wearer's body. The three-dimensional capture structure 80 is adapted to fit at least partially into a wearer's gluteal fold when the absorbent pad 20 is placed in a wearer's undergarment and positioned in the wearer's perianal region. As used herein, the term “gluteal fold” refers generally to the space that naturally forms between the buttocks. Also, as used herein, the term “perianal” means that region of the body generally between the anus and the posterior part of the external genitalia and the region generally around the anus. As used herein, the term “adapted to fit into a wearer's gluteal fold” refers to positioning a structure on an absorbent pad such that the structure resides proximate a wearer's gluteal fold and fits at least partially within the wearer's gluteal fold and dimensioning the structure so as to approximate the general shape and size of the gluteal fold and thus reduce the area through which fluid may pass when the absorbent pad 20 is placed in the wearer's undergarment and positioned in the wearer's perianal region. For example, FIG. 8 representatively illustrates a three-dimensional capture structure 80 positioned on an absorbent pad 20 adapted to fit into a wearer's gluteal fold.

In some embodiments, instructions may be provided to a wearer or caregiver on the proper positioning and use of the present invention. The instructions may include information enabling the wearer to properly position the absorbent pad 20 in the wearer's undergarment. The instructions may further describe the function of the three-dimensional capture structure 80 and the improved protection from rear leakage if the absorbent pad 20 is positioned in the perianal region and the three-dimensional capture structure 80 is positioned at least partially within the wearer's gluteal fold. The instructions may further assist a wearer to position the three-dimensional capture structure 80 in the rear of the absorbent pad 20 and not the front. The instructions may also inform a wearer that placing the absorbent pad 20 with the three-dimensional capture structure 80 in the front may reduce or eliminate the leakage benefits possible with the three-dimensional capture structure 80 positioned in the rear. A wearer may reduce rear leakage by following the instructions provided in combination with use of the absorbent pad 20 of the present invention.

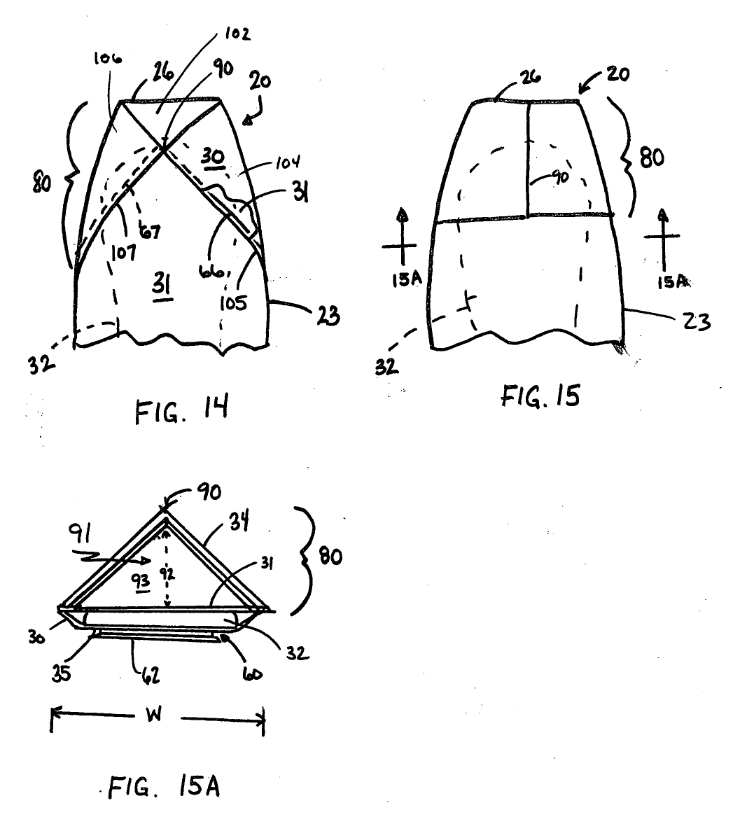

In various embodiments, the three-dimensional capture structure 80 may be a unitary structure formed by extended portions of the backsheet layer 30, the topsheet layer 31, the side panel members 42 and 44, or combinations thereof. In alternative embodiments, the three-dimensional capture structure 80 may be a non-unitary structure formed partially or completely by additional separate and distinct elements bonded to the absorbent pad 20. As used herein, the term “unitary structure” means a three-dimensional capture structure 80 constructed primarily of either an uninterrupted, continuous or unbroken backsheet layer 30, topsheet layer 31, side panel members 42 and 44, or combinations thereof. It is contemplated that unitary structures can include additional elements such as adhesive or other bonding means and elastic components. For example, FIG. 1 representatively illustrates an absorbent pad 20 with a unitary three-dimensional capture structure 80. As used herein, the term “non-unitary structure” refers to a three-dimensional capture structure constructed of material or materials separate or discrete from the topsheet or backsheet layers. For example, a capture structure constructed of separate (i.e., discrete, discontinuous) but identical pieces of the top sheet or backsheet layers would be a non-unitary structure. For example, FIG. 16 representatively illustrates an absorbent pad 20 with a non-unitary three-dimensional capture structure 80.

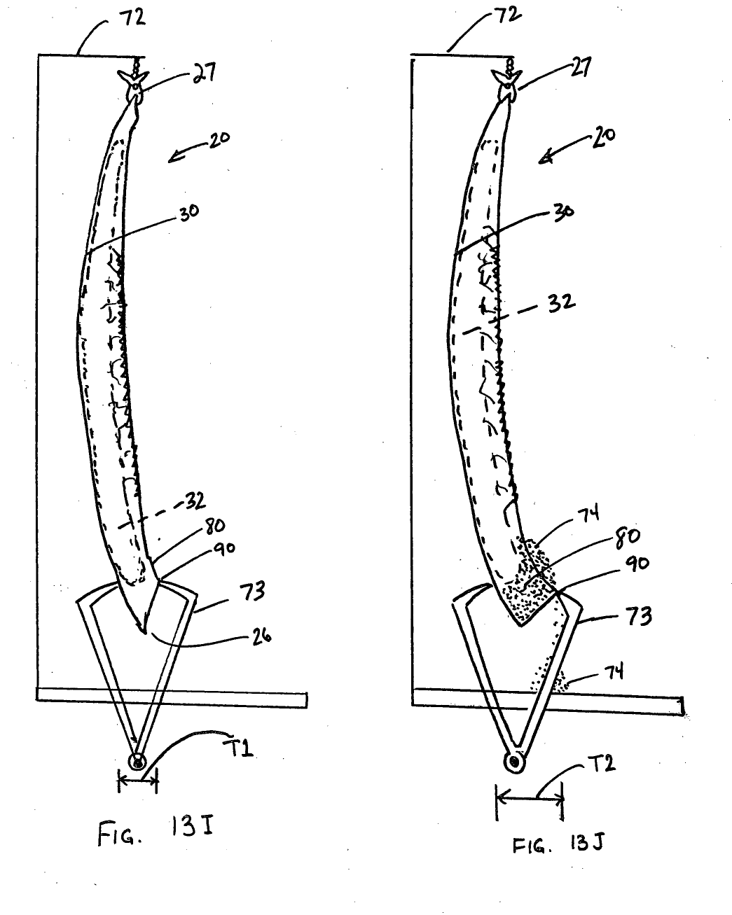

FIG. 3A representatively illustrates a transverse section view of the rear portion 23 of the absorbent pad 20 of FIG. 1 taken along the line 3A-3A. The embodiment illustrated in FIG. 3A is a unitary structure in that the three-dimensional capture structure 80 is constructed using extended portions of the backsheet layer 30 and the topsheet layer 31 to form the three-dimensional capture structure 80, including panels 102, 104, and 106 peaking at an apex 90. The panel 104 has a free edge 105 and the panel 106 has a free edge 107. The free edges 105 and 107 may join at the apex 90. The apex 90 is generally centered on the transverse axis 24 and is located atop an entrance 91 (shaded area of FIG. 3A) of the three-dimensional capture structure 80. The entrance 91 is defined by free edges 105 and 107 and the topsheet layer 31. The entrance 91 also has a height 92. The panels 102,104,106, entrance 91 and the topsheet layer 31 define a volume 93. The volume 93 is generally the volume available within the three-dimensional capture structure 80 to capture and retain exudates. FIG. 15 representatively illustrates the rear portion 23 of an exemplary absorbent pad 20 with a three-dimensional capture structure 80. FIG. 15A representatively illustrates a cross sectional view of the absorbent pad 20 of FIG. 15 taken along line 15A-1 5A. FIG. 15A illustrates the entrance 91, the height 92, and the volume 93 of the three-dimensional capture structure 80.

In general, the three-dimensional capture structure 80 captures and retains body exudates that may otherwise leak from the longitudinal end edge 26 of the absorbent pad 20. The apex 90 is generally centered upon the wearer's body in the gluteal groove when the absorbent pad 20 is positioned in an undergarment adjacent a wearer's perianal region as representatively illustrated in FIG. 8 (undergarment not shown). Referring again to FIG. 3A, urine and other exudates that are not absorbed into the absorbent assembly 32 may move along the topsheet 31 towards the rear portion 23 and pass through the entrance 91 into the volume 93 of the three-dimensional capture structure 80. In embodiments involving a unitary structure, the three-dimensional capture structure 80 may be made liquid impervious either through use of a liquid impervious backsheet layer 30, or treating the topsheet layer 31, or combinations thereof. In embodiments involving a unitary structure, the three-dimensional capture structure 80 may be elasticized either through use of a backsheet layer 30 made of elastomeric materials, or a topsheet 31 made of elastomeric materials, or combinations thereof. In embodiments utilizing a non-unitary structure, the three-dimensional capture structure 80 may be made liquid impervious by adding liquid impervious materials and creating a liquid impervious bond between the three-dimensional capture structure 80 and the absorbent pad 20. A liquid impervious bond can be created with adhesive, ultrasonic bonding, thermal bonding, or other suitable attachment means known in the art. In embodiments utilizing a non-unitary structure, the three-dimensional capture structure 80 may be elasticized by adding elastomeric material or materials. When the three-dimensional capture structure 80 is made liquid impervious, captured exudate is prevented from soiling the wearer's undergarments and may eventually be absorbed by the absorbent assembly 32. When the three-dimensional capture structure 80 is elasticized, the volume 93 may increase as the three-dimensional capture structure 80 captures and retains bodily exudate.

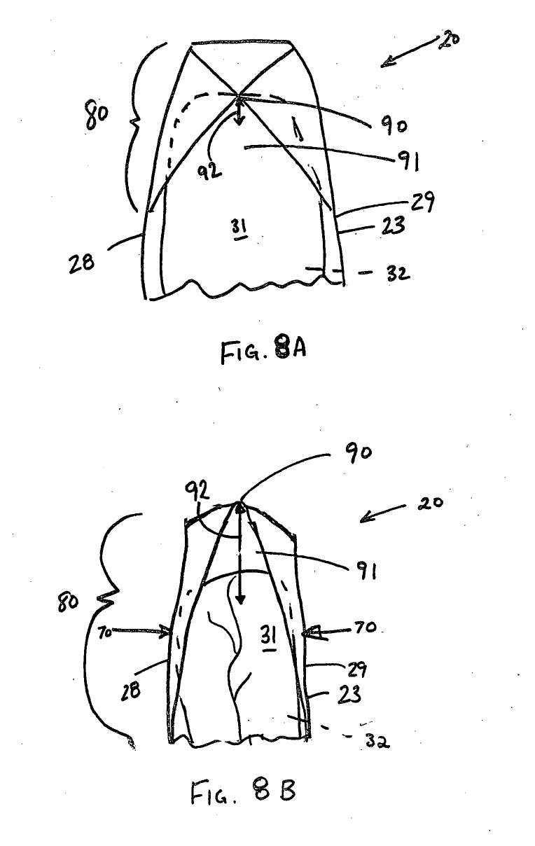

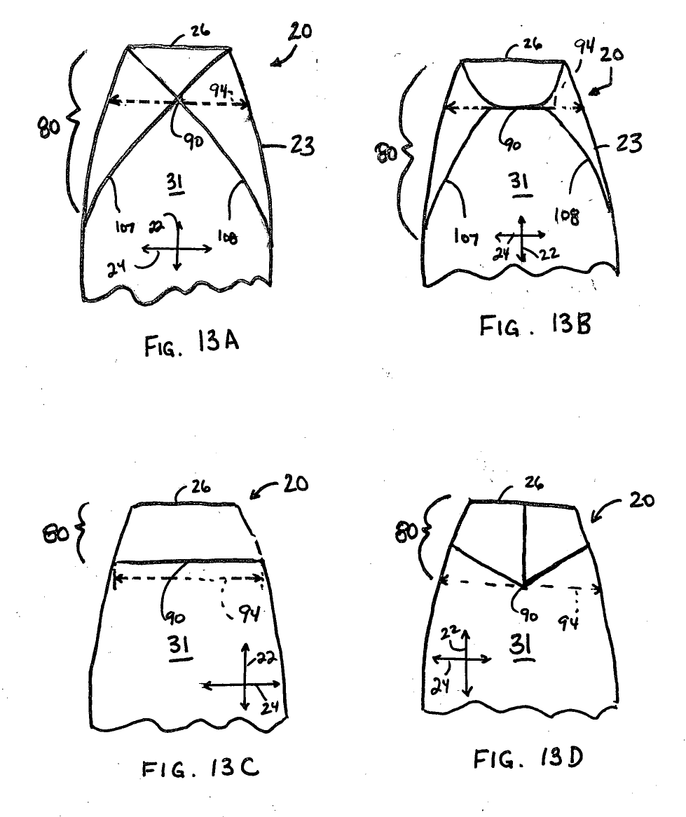

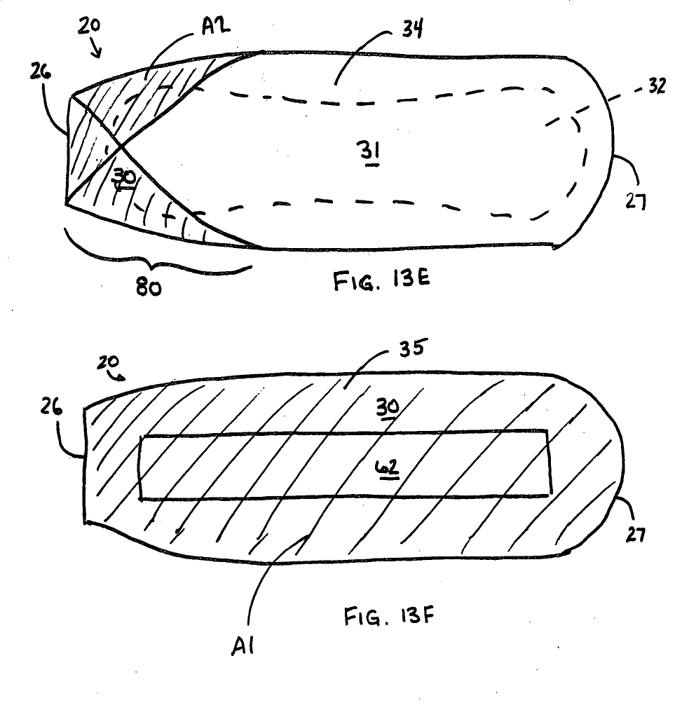

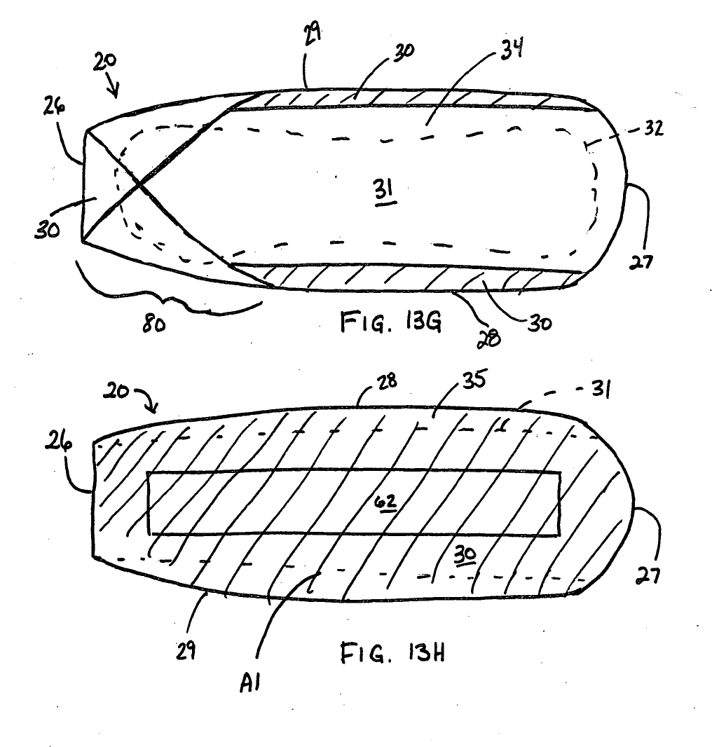

In various embodiments, the entrance 91 has a generally triangular shape as a result of the generally pyramidal shape of the three-dimensional capture structure 80, although other suitable shapes are also conceived. For example, the entrance 91 may be a pentagon as generally shown in FIG. 3A. The entrance 91 may also result in a generally flat portion at the apex 90 as illustrated in FIG. 13B. The specific shape of the three-dimensional capture structure 80 and the entrance 91 need only be adapted to fit in close proximity with the wearer's gluteal groove and provide access to the volume 93 of the three-dimensional capture structure 80. The embodiment illustrated in FIGS. 1, 2, and 3A have a three-dimensional capture structure 80 with a generally pyramidal shape. It is believed that the anatomical coordination between the three-dimensional capture structure 80 and the gluteal groove decreases the area through which rear leakage can occur as illustrated in FIG. 8. In contrast, FIG. 9 representatively illustrates an absorbent pad without a three-dimensional capture structure in the rear of the pad. The absorbent pad of FIG. 9 has no means of preventing exudate moving along the top surface of the absorbent pad from leaking at the rear of the pad, particularly along the gluteal groove. The generally pyramidal shape of the three-dimensional capture structure 80 is believed to coordinate well with the gluteal groove and thus reduce the amount of fluid that may pass between the three-dimensional capture structure 80 and the gluteal groove. The generally pyramidal shape of the three-dimensional capture structure 80 may also decrease the amount of crushing that occurs during use as a result of sitting or lying on the absorbent pad 20. As a wearer sits or lies down, pressure is applied to the buttocks and the absorbent pad 20 as a result. But the gluteal groove provides a recess in which the three-dimensional capture structure 80 can reside with minimal crushing, thus allowing fluid to pass through the entrance 91 and into the volume 93.

In various embodiments, the three-dimensional capture structure 80 is adapted to work in coordination with the side retention members 40. As discussed previously, fluids that run along the topsheet 31 can leak from the sides and rear of the absorbent pad 20. The side retention members 40 can reduce leakage from the sides, but fluid contacting side retention members 40 may flow along the side retention members 40 and towards the rear portion 23 of the absorbent pad 20. As can be seen in FIGS. 1, 2, and 3A, the side retention members 40 form a smooth and continuous transition with the three-dimensional capture structure 80 ensuring fluid flowing along side retention members 40 does not escape the absorbent pad 20, but is directed into the three-dimensional capture structure 80 where the fluid is captured, retained and/or absorbed. In embodiments wherein the side retention members 40 contain elastic members 46, the interaction with the three-dimensional capture structure 80 can be improved. As previously mentioned, the elastic members 46 and the side retention members 40 extend above the plane of the absorbent assembly 32, creating a bucket shaped structure in which the fluid can be retained until it is absorbed by the absorbent pad 20. The elastic members 46 provide tension to the free edge 105 of the panel 104 and the free edge 107 of the panel 106, increasing the area of the entrance 91 and the volume 93 of the three-dimensional capture structure 80. Tension around the entrance 91 provides resistance against closing of the entrance 91 during use and resistance against collapsing of the three-dimensional capture structure 80.

Tension around the entrance 91 may also be provided, in various embodiments, by adding elastic to the three-dimensional capture structure 80. Referring now to FIG. 14, some embodiments of the present invention may include a capture elastic 66 disposed generally parallel to the free edge 105 of the panel 104 and a capture elastic 67 disposed generally parallel to the free edge 107 of the panel 106. In such embodiments, the capture elastics 66 and 67 may be disposed and operatively joined to the topsheet layer 31, to the backsheet layer 30, or sandwiched between the topsheet layer 31 and the backsheet layer 30 as illustrated. The capture elastics 66 and 67 can be applied under tension resulting in at least partial contraction along the free edges 105 and 107. The capture elastics 66 and 67 may be bonded to the three-dimensional capture structure 80 in a stretched condition, bonded in a relaxed state to a gathered portion of the three-dimensional capture structure 80, or may have elastic properties activated after being bonded to the three-dimensional capture structure 80. The forces created by the capture elastics 66 and 67 in the free edges 105 and 107 help open the entrance 91, and, as a consequence, the volume 93 is available to capture exudates in the three-dimensional capture structure 80. The contraction and tension provided by the capture elastics 66 and 67 also helps retain the height 92 of the three-dimensional capture structure 80 during use. It is believed that retention of the height 92 will allow the three-dimensional capture structure 80 to more completely fill the gluteal groove and thus provide more effective reduction of fluid leakage at the rear of the absorbent pad 20.

The capture elastics 66 and 67 may be formed of one or more dry-spun coalesced multifilament elastomeric threads sold under the tradename LYCRA and available from E.I. Du Pont de Nemours and Company. Alternately, the capture elastics 66 and 67 may be formed of other typical elastics utilized in making incontinence products, such as a thin ribbon of natural rubber, wet-spun spandex materials, a stretch bonded laminate material comprising a prestretched elastic meltblown inner layer sandwiched between and bonded to a pair of spunbond polypropylene nonwoven webs, or the like. Elasticity could also be imparted to the three-dimensional capture structure 80 by extruding a hot melt elastomeric adhesive along or proximate the free edges 105 and 107. Elasticity could also be imparted to the three-dimensional capture structure 80 by use of a backsheet 30 made of elastomeric materials, a topsheet 31 made of elastomeric materials or combinations thereof for construction of the three-dimensional capture structure 80 as discussed above. It is believed that use of elastomeric materials in the three-dimensional capture structure 80 helps to retain the height 92 of the three-dimensional capture structure 80 during use. It is believed that retention of the height 92 will allow the three-dimensional capture structure 80 to more completely fill the gluteal groove and thus provide more effective reduction of fluid leakage at the rear of the absorbent pad 20. It is also believed that use of elastomeric materials in the three-dimensional capture structure 80 helps maintain contact between the three-dimensional capture structure 80 and the perianal region of the wearer while moving. The elasticized three-dimensional capture structure 80 is believed to move with the wearer and stretch to a certain degree before becoming disengaged from the gluteal groove.

In various embodiments, the entrance 91 to the three-dimensional capture structure 80 disposed on the absorbent pad 20 may also be opened through the use of compressive forces produced between a wearer's legs. FIG. 8A illustrates a rear portion 23 of an absorbent pad 20 according to the present invention with three-dimensional capture structure 80 disposed in the rear portion 23 on the body facing surface 34. The embodiment illustrated in FIG. 8A is shown in a relaxed and laid flat position. The height 92 of the three-dimensional capture structure 80 is relatively small as compared to the height 92 of the three-dimensional capture structure 80 as illustrated in FIG. 8B. FIG. 8B representatively illustrates the absorbent pad 20 of FIG. 8A after an external force is applied to the longitudinal side edges 28 and 29 in a direction generally indicated by arrows 70. Such force is applied by the legs of a wearer after placing the absorbent pad 20 in an undergarment and positioning the undergarment and absorbent pad 20 in the perianal region. As is illustrated in FIG. 8B, it is believed that compressive forces applied in the direction 70 causes the apex 90 to move in the z-direction away from the topsheet layer 31, thus increasing the height 92 and opening the entrance 91 of the three-dimensional capture structure 80. In use, it is advantageous for the three-dimensional capture structure 80 to have an open entrance 91 so as to more completely fill the gluteal groove and reduce leakage that may occur from the rear portion 23 of the absorbent pad 20. In this way, the absorbent pad 20 and the three-dimensional capture structure 80 are adapted to be positioned such that the three-dimensional capture structure 80 is aligned with and proximate the gluteal groove.

The three-dimensional capture structure 80 has a height 92, measured in the z-direction from the apex 90 to the topsheet 31. The height 92 is measured by hanging the absorbent pad 20 in a vertical position as illustrated in FIG. 13I. The pad 20 is hung from a support 72 with the three-dimensional capture structure 80 down. A caliper measuring device 73 is used to determine a collapsed thickness, T1, of the absorbent article 20 at the apex 90. Only enough pressure is exerted during measurement to close or collapse the three-dimensional capture structure 80 while preventing compression of the absorbent assembly 32 as illustrated in FIG. 131. In various embodiments, the absorbent assembly 32 may not extend into the three-dimensional capture structure 80. In such situations, the collapsed thickness, T1, will not include the thickness of the absorbent assembly 32. A particulate 74 having relatively small particle size such as sugar, salt, or fine sand is added to fill the volume 93 of the three-dimensional capture structure 80 until the particulate 74 spills from the three-dimensional capture structure 80. The caliper measuring device 73 is then used to determine a thickness, T2, of the filled three-dimensional capture structure 80 at the apex 90 as illustrated in FIG. 13J. As mentioned above, in some embodiments, the absorbent assembly 32 may not extend into the three-dimensional capture structure 80 and the filled thickness, T2, will not include the thickness of the absorbent assembly 32. Whether, the absorbent assembly 32 extends into the three-dimensional capture structure 80 or not, the height 92 is calculated by subtracting the collapsed thickness, T1, from the filled thickness, T2. In various embodiments, the height 92 can be from about 5 mm to about 75 mm, from about 10 mm to about 60 mm, from about 15 mm to about 45 mm, from about 20 mm to about 30 mm, or about 25 mm.

The three-dimensional capture structure 80 also has a width 94 measured in the direction of the transverse axis 24. The width 94 is measured by placing the absorbent pad 20 with the body-facing surface 34 up and in a laid flat and relaxed position. The width 94 is measured along the transverse axis 24 at the apex 90. FIGS. 13 A-D illustrate examples of different three-dimensional capture structures 80 and illustrate the location at which the width 94 is measured. In various embodiments, the width 94 can be from about 10 mm to about 230 mm, from about 20 mm to about 200 mm, from about 30 mm to about 150 mm, from about 40 mm to about 100 mm, from about 50 mm to about 90 mm, from about 60 mm to about 80 mm, or from about 65 mm to about 70 mm.

The width 94 and the height 92 of the three-dimensional capture structure 80 are adapted to cooperate with a wearer's gluteal fold. The width 94 and the height 92 are also adapted to at least partially fit within the gluteal groove. This is desirable because the three-dimensional capture structure 80 may have a large volume 93 prior to use, but will be crushed upon use if the three-dimensional capture structure 80 is not at least partially positioned within the gluteal groove. For example, a very wide three-dimensional capture structure 80 may have a large volume 93 but may not fit within the gluteal groove. In such a scenario, the compressive force applied during use would likely crush the three-dimensional capture structure 80 and reduce or eliminate the volume 93. Conversely, a very narrow three-dimensional capture structure 80 may be well suited for use with “thong” style undergarments wherein engagement with the gluteal groove is facilitated by the design of the absorbent pad 20. In another example, the three-dimensional capture structure 80 may have a large volume 93 because of a large height 92. A large height 92 may allow the three-dimensional capture structure 80 to remain engaged within the gluteal groove as the absorbent pad 20 moves towards and away from the wearer as may occur when the wearer sits, stands, or walks.

Therefore, the ratio of the width 94 to the height 92 must be adapted to be wide enough and high enough to create adequate volume 93 but not so high or so wide that the three-dimensional capture structure 80 is crushed upon use. In various embodiments, the ratio of the width 94 to the height 92 can be less than about 4 to 1. For example, the width 94 may be about 80 mm and the height 92 may be about 20 mm. At ratios higher than 4 to 1, it is believed that the three-dimensional capture structure 80 would be generally wide and flat and may provide adequate volume 93, but may not adequately engage in the gluteal fold of the wearer. As such, the three-dimensional capture structure 80 would likely be crushed and rendered ineffective when the wearer sat or otherwise applied compressive forces to the absorbent pad 20. However, ratios less than about 4 to 1 are believed to be more effective in reducing rear leakage. For example, in some embodiments, the ratio of the width 94 to the height 92 may be about 3 to 1. For example, the width 94 may be about 75 mm and the height 92 may be about 25 mm. In yet other embodiments, the ratio of the width 94 to the height 92 may be about 2 to 1. For example, the width 94 may be about 70 mm and the height 92 may be about 35 mm. In yet other embodiments, the ratio of the width 94 to the height 92 may be about 1 to 1. For example, the width 94 may be about 35 mm and the height 92 may be about 35 mm. In yet other embodiments, the ratio of the width 94 to the height 92 may be about 1 to 2. For example, the width 94 may be about 10 mm and the height 92 may be about 20 mm. In yet other embodiments, the ratio of the width 94 to the height 92 may be about 1 to 3. For example, the width 94 may be about 10 mm and the height 92 may be about 30 mm. In yet other embodiments, the ratio of the width 94 to the height 92 may be about 1 to 4. For example, the width 94 may be about 10 mm and the height 92 may be about 40 mm.

Referring to FIGS. 6 and 7, an absorbent pad 20 is shown in interim steps during the formation of a three-dimensional capture structure 80, as illustrated in FIG. 4. The three-dimensional capture structure 80 is constructed by both the backsheet layer 30 and the topsheet layer 31 creating a unitary structure. In such embodiments, the backsheet layer 30 or the topsheet layer 31 or both are manufactured to a length along the longitudinal axis 22 greater than the desired finished length to allow construction of a unitary three-dimensional capture structure 80. In the embodiment of FIGS. 6 and 7, the backsheet layer 30 and the topsheet layer 31 have a temporary longitudinal end edge 126. To construct this embodiment, a bonding means is applied generally in a bonding area 98 and then the temporary longitudinal end edge 126 is folded in the direction indicated by an arrow 83 towards the body-facing surface 34 of the absorbent pad 20 along a fold line 82 forming a flap 96 and the longitudinal end edge 26. Alternatively, the bonding can occur in the bonding areas 98 after folding the temporary longitudinal end edge 126. The bonding means applied in the bonding areas 98 can be adhesives, mechanical bonds, heat bonds, ultrasonic bonds, or any other suitable bonding means and combinations thereof. After the flap 96 is formed, a bonding means is applied generally to the bonding areas 100. The absorbent pad 20 is folded along fold lines 84 in a direction indicated by the arrows 86 thus forming the panels 102, 104, 106, the entrance 91, and the volume 93 as illustrated in FIG. 3A. Alternatively, the bonding in the bonding areas 100 can occur after the three-dimensional capture structure 80 is formed.

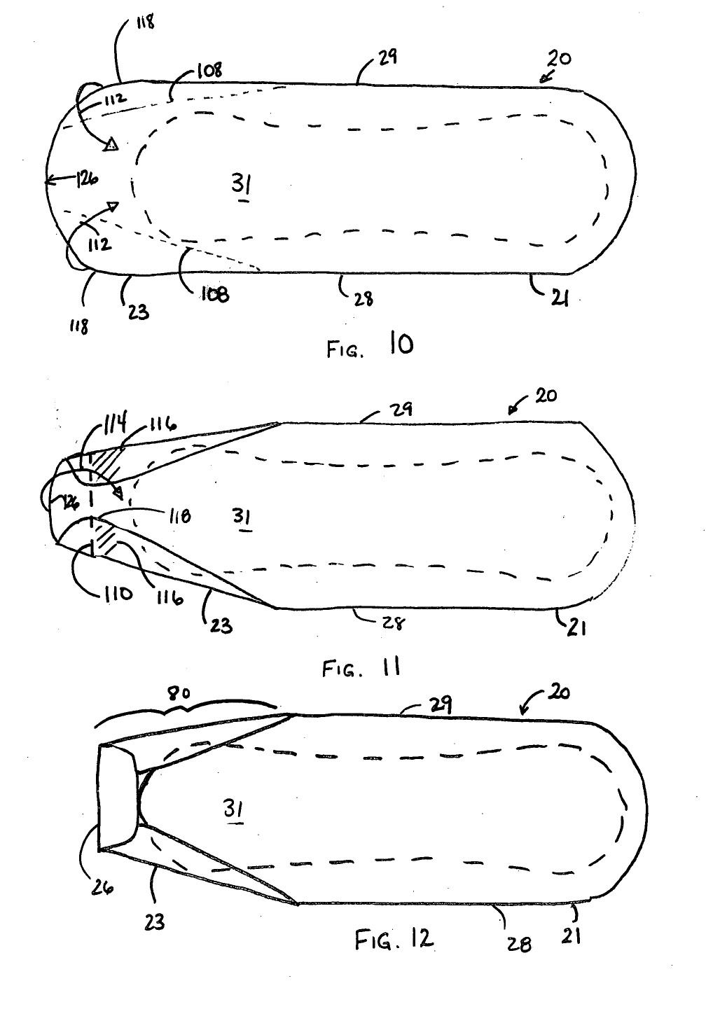

Referring now to FIGS. 10, 11, and 12, alternate folding can be used to create an absorbent pad 20 with a unitary three-dimensional capture structure 80. For example, the absorbent pad 20 may have corners 118 located at the junctions of the longitudinal side edges 28 and 29 and the temporary longitudinal end edge 126 located in the rear portion 23. The corners 118 are folded towards the topsheet layer 31 along the fold lines 108 in a direction indicated by arrows 112. Adhesive or other bonding means are applied generally in bonding areas 116. The temporary longitudinal end edge 126 is then folded towards the topsheet layer 31 along the fold line 110 in the direction indicated by an arrow 114 forming the longitudinal end edge 26 and the three-dimensional capture structure 80. Alternatively, the bonding in the bonding areas 116 can occur after the temporary longitudinal end edge 126 is folded.

FIG. 13E representatively illustrates one embodiment of an absorbent pad 20 according to the present invention. The absorbent pad 20 of FIG. 13E is shown in a laid flat and relaxed configuration with the body-facing surface 34 up. FIG. 13F representatively illustrates the absorbent pad 20 of FIG. 13E in a laid flat and relaxed position with the garment-facing surface 35 up. In unitary embodiments wherein the three-dimensional capture structure 80 is constructed of the backsheet layer 30, a portion of the backsheet layer 30 is disposed on the garment-facing surface 35 and has a garment facing area, A1, as illustrated by hatch marks in FIG. 13F. Also, a portion of the backsheet layer 30 is disposed on the body-facing surface 34 and has a body-facing area, A2, as representatively illustrated by hatch marks in FIG. 13E. The area, A1, is measured while the absorbent pad 20 is in a laid flat and relaxed condition with the garment-facing surface 35 facing up. The area, A2, is measured while the absorbent pad 20 is in a laid flat condition with the body-facing surface 34 facing up. In some embodiments, the backsheet layer 30 may be wider than the topsheet layer 31 as representatively illustrated in FIGS. 13G and 13H. FIG. 13G representatively illustrates one embodiment of the present invention showing an absorbent pad 20 comprising a backsheet layer 30 that is wider than a topsheet layer 31. The absorbent pad 20 of FIG. 13G is shown in a laid flat condition with the body-facing surface 34 up. FIG. 13H representatively illustrates the absorbent pad 20 of FIG. 13G shown in a laid flat condition with the garment-facing surface 35 up. As illustrated, the longitudinal side edges 28 and 29 are composed only of the backsheet layer 30. As such, the backsheet layer 30 is exposed on the body-facing surface 34, as representatively illustrated by hatch marks in FIG. 13G, in areas other than the three-dimensional capture structure 80. This area is not included in the calculation of the area, A2. However, this area is still included in the calculation of the area, A1, as representatively illustrated by hatch marks in FIG. 13H.

In various embodiments, the ratio of A1/A2 is less than about 7 to 1. For example, A1 may be about 260 cm² and A2 may be about 40 cm². In other embodiments, the ratio of A1/A2 is less than about 6 to 1. For example, A1 may be about 260 cm² and A2 may be about 45 cm². In yet other embodiments, the ratio of A1/A2 is less than about 5 to 1. For example, A1 may be about 260 cm² and A2 may be about 52 cm². In yet other embodiments, the ratio of A1/A2 is less than about 4 to 1. For example, A1 may be about 260 cm² and A2 may be about 65 cm².

As previously discussed, the body-facing surface 34 is made of a topsheet layer 31 and the three-dimensional capture structure 80. The body-facing surface 34 of the three-dimensional capture structure 80 may include the backsheet layer 30. Therefore, the body-facing surface 34 may include a topsheet layer 31 and a portion of the backsheet layer 30. It is believed that the area, A2, generally corresponds to the volume 93 of the three-dimensional capture structure 80. Therefore, if the area A2 increases, the volume 93 will generally increase as well. The volume 93 corresponds to the quantity of bodily exudate that can be captured and/or retained by the three-dimensional capture structure 80. In embodiments having a higher A1/A2 ratio, such as 7 to 1 or 6 to 1, the body-facing surface 34 has relatively more of the topsheet layer 31 exposed than is covered by the liquid impermeable backsheet layer 30. As such, a higher ratio of A1/A2 means there is more area available on the body-facing surface 34 to receive urine or exudate insults. While it is advantageous to have more absorbent surface available, there are also advantages to a larger three-dimensional capture structure 80 and associated larger area A2, as discussed above. In general, a larger three-dimensional capture structure 80 provides greater exudate capture and retention in the rear portion 23 of absorbent pad 20. As the area of backsheet layer 30 increases on the body-facing surface 34 as a consequence of a larger three-dimensional capture structure 80, the ratio A1/A2 decreases. In embodiments having a smaller A1/A2 ratio, such as 5 to 1 or 4 to 1, the body-facing surface 34 is more covered with the backsheet layer 30 and less of the topsheet layer 31 is exposed. As such, the body-facing surface 34 available to receive urine or other exudate discharges is diminished. Therefore, it is important to achieve a proper balance between three-dimensional capture structure size and the area, A2, of the body-facing surface 34 covered by the backsheet layer 30. While the present invention contemplates A1/A2 ratios as low as 2 to 1 for maximum rear protection, ratios ranging from about 7 to 1 to about 4 to 1 are believed to better balance the dual objectives of maximizing the area available to receive insults and minimizing rear leakage.

In yet other embodiments, one or more separate pieces of material may be added to the absorbent pad 20 to construct a non-unitary three-dimensional capture structure 80. FIG. 16 representatively illustrates an absorbent pad 20 with a three-dimensional capture structure 80 disposed on the body-facing surface 34 and comprising additional pieces of material. FIG. 17 representatively illustrates a cross-sectional view of a portion of the absorbent pad 20 of FIG. 16, taken along line 17-17.

In FIGS. 16 and 17, the illustrated three-dimensional capture structure 80 is elasticized and is connected to at least one of the backsheet layer 30 and the topsheet layer 31 along or near the end margin 26 of the absorbent pad 20. The three-dimensional capture structure 80 is connected along a region of attachment 226. The means of attachment in the region of attachment 226 can be mechanical, adhesive, ultrasonic, thermal, chemical, or other suitable attachment means, or combinations thereof. The shown three-dimensional capture structure 80 includes an extending flange section 182 and an extending three-dimensional section 184. The three-dimensional section 184 of the three-dimensional capture structure 80 includes a substantially fixed edge portion 202 secured to the absorbent pad 20, and includes an elasticized, gathered moveable edge portion 204, which is longitudinally spaced from the fixed edge portion 202. The three-dimensional capture structure 80 also includes a substantially liquid impermeable barrier layer 206, and a fabric layer 208 connected in facing relation with the barrier layer 206. A plurality of separate, laterally extending elastic members 210 are sandwiched between the barrier layer 206 and the fabric layer 208 to provide an elasticized three-dimensional composite 212 which is substantially laterally gathered. A plurality of separate, laterally extending flange elastic members 218 are also sandwiched between the barrier layer 206 and the fabric layer 208 in the flange section 182.

The fabric layer 208 may, for example, be composed of a woven or nonwoven fabric, and in the shown arrangement, the fabric layer 208 is desirably a nonwoven. A plurality of separate, laterally extending elastic members 210 are sandwiched and operably connected between the barrier layer 206 and the fabric layer 208 to provide an elasticized three-dimensional composite 212, which is gathered substantially along the transverse axis direction 24 and is elastically stretchable at least along the transverse axis direction 24. The shown arrangement includes elastic members which are aligned substantially parallel to one another, but optionally can include other separated configurations and alignments of the elastics. Desirably, the fabric layer 208 is arranged for placement against the wearer's skin, although the barrier layer 206 may optionally be appointed for placement immediately adjacent the wearer's skin.

In regard to either or both of the flange section 182 and the extending three-dimensional section 184 of the three-dimensional capture structure 80, the barrier layer 206 can be provided by polymer films or fabrics having low permeability to liquid, and combinations thereof. Polymer films may, for example, be composed of polyolefins, polyesters, polyamides and the like. Nonwoven materials can include spunbond-meltblown-spunbond (SMS) fabrics, meltblown fabrics, calendered nonwoven sheets and the like. With respect to the passage of liquid through its thickness, the barrier layer 206 is constructed to exhibit a hydrohead of resistance which is sufficient to provide an effective barrier against the passage liquids, such as urine. For example, the barrier layer 206 may be composed of a 0.0006 inch (0.015 mm) cast, embossed film, such as a CT (XEM400.1), or a 0.0004 inch (0.010 mm) blown film, such as XSF-367, available from Consolidated Thermoplastics, a business having offices located in Chippewa Falls, Wis. The barrier layer 206 may also be a 0.00035 inch (0.0089 mm) stretch-thinned film, such as XP1024A, available from Edison Plastics, a business having offices located in Macalester, Okla.

With regard to either or both of the flange section 182 and three-dimensional section 184 of the three-dimensional capture structure 80, the fabric layer 208 can be composed of a fine denier, low basis weight nonwoven material. Examples of such nonwoven fabrics include polypropylene spunbond materials, bicomponent polypropylene/polyethylene spunbond materials, meltblown materials, SMS materials, through-air-bonded carded webs, point-bonded bonded-carded webs, and the like. For example, the fabric layer 208 may be a 0.5 osy (17 gsm) polypropylene spunbond fabric composed of fibers having denier of less than about 4 denier. The fabric layer 208 can alternatively have fibers with deniers of less than about 3 denier, and optionally can include fibers having deniers of less than about 2.5 denier.

Either or both of the elastic members 210 and the flange elastic members 218 can be composed of strands of natural or synthetic elastomeric materials, such as natural or synthetic rubbers. In particular aspects of the invention, the elastic members can include strands having a denier of not less than about 100 denier. Alternatively, the elastic members can have a denier of not less than about 280, and optionally can have a denier of not less than about 360. In other aspects of the invention, the elastic members can include strands having a denier of not more than about 1920 denier. Alternatively, the elastic members can have a denier of not more than about 1140 denier, and optionally can have a denier of not more than about 560 denier. For example, the elastic members 210 and/or the flange elastic members 218 can include 360 denier GLOSPAN S7 elastic strands available from Radici Spandex Corporation, having offices at 125 Hartwell Street, Fall River, Mass. 02721.

In various other embodiments, additional materials may be added to the absorbent pad 20 to create a three-dimensional capture structure 80. In such embodiments, the three-dimensional capture structure 80 may or may not be elasticized. Other techniques for constructing a structure suitable for use as the three-dimensional capture structure 80 in the present invention are disclosed in U.S. Pat. No. 5,827,259 to Laux et al. issued Oct. 27, 1998, the entirety of which is incorporated herein by reference.

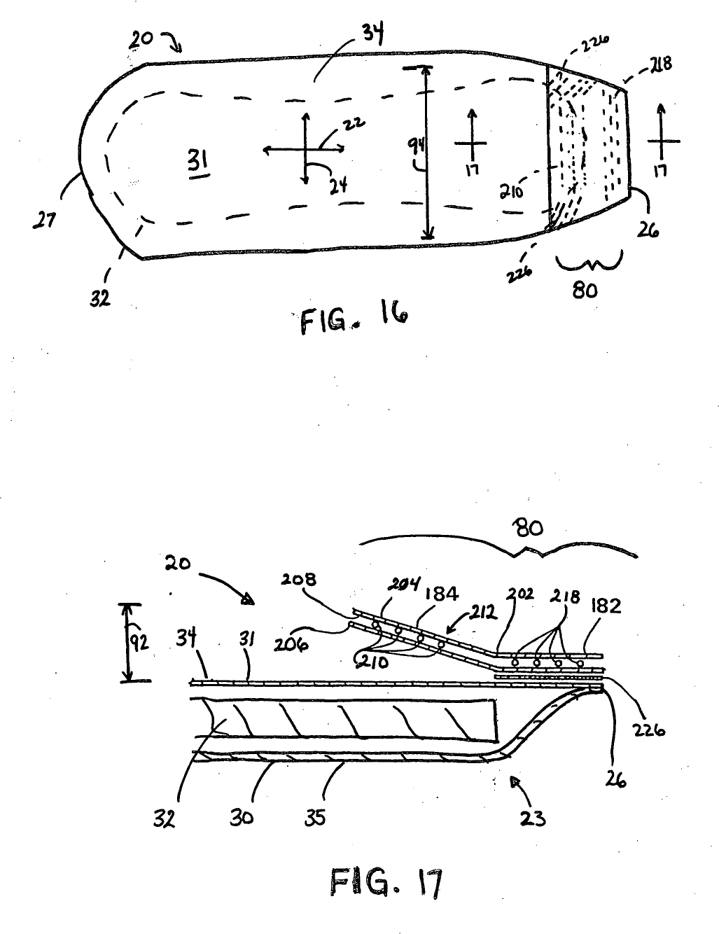

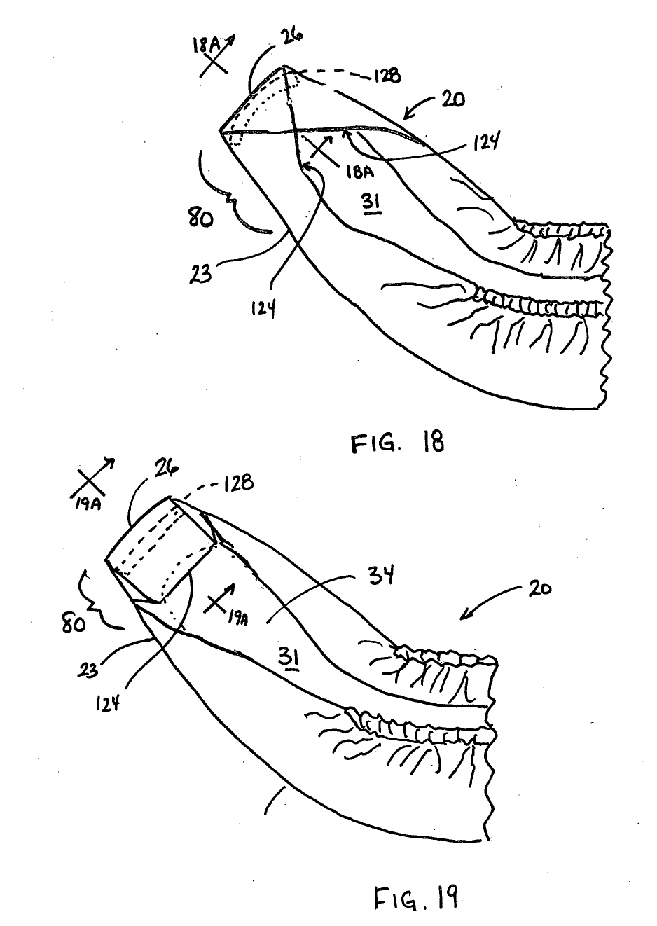

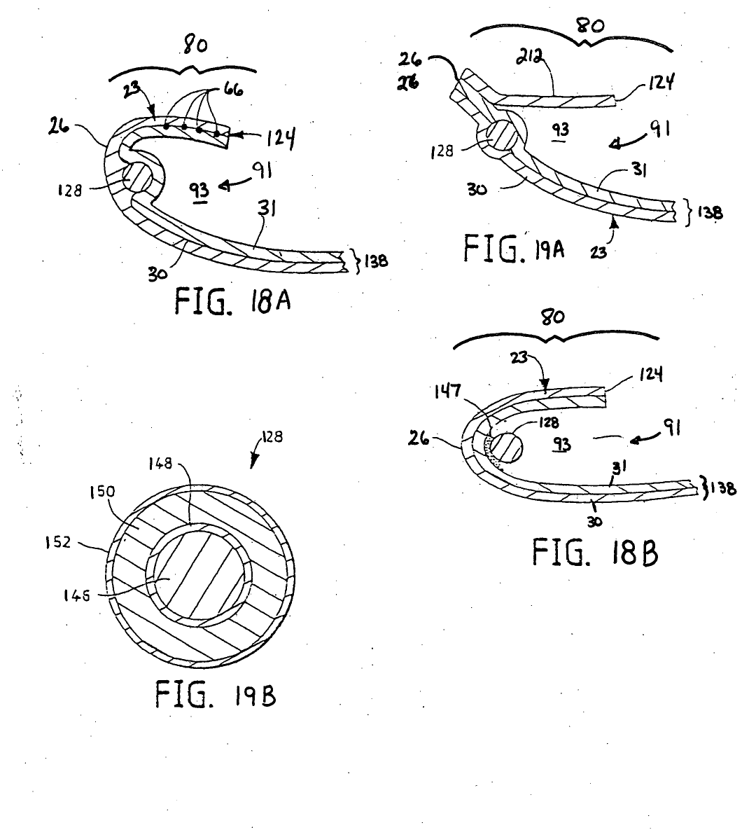

In various embodiments, the absorbent pad 20 may include a spacer 128 operably associated with the three-dimensional capture structure 80 as representatively illustrated in FIGS. 18 and 19. The absorbent article 20 may include a three-dimensional capture structure 80 having at least one distal edge 124. In various embodiments, the three-dimensional capture structure 80 can be a folded over length of the rear portion 23 as representatively illustrated in FIG. 18. In other embodiments, the three-dimensional capture structure 80 can include additional materials attached to rear portion 23 of absorbent pad 20 as representatively illustrated in FIG. 19. The three-dimensional capture structure 80 may be attached with glue or other suitable bonding means such as, for example, sewing, heat sealing, sonic bonding, adhesive bonding, or the like.

The spacer 128 may be mounted in the rear portion 23 proximate the longitudinal end edge 26. The spacer 128 works in combination with the three-dimensional capture structure 80 to protect against leakage of exudates from the rear portion 23 of the absorbent pad 20. FIG. 18A illustrates a cross-sectional view of the rear portion 23 taken at line 18A-18A of FIG. 18. FIG. 18A shows a base structure 138 formed by a backsheet layer 30 and a topsheet layer 31. The spacer 128, attached to the base structure 138, helps open the entrance 91 and form the volume 93. In this embodiment, the unitary three-dimensional capture structure 80 is constructed of a folded over length of the rear portion 23 and in various embodiments may include capture elastics 66. The spacer 128 preferably is glued or otherwise attached to the base structure 138 between the backsheet layer 30 and the topsheet layer 31 as representatively illustrated in FIG. 18A.

FIG. 19 representatively illustrates an embodiment wherein a non-unitary three-dimensional capture structure 80 is constructed of additional materials attached to rear portion 23 of absorbent pad 20. The three-dimensional capture structure 80 may be attached with glue or other suitable bonding means such as, for example, sewing, heat sealing, sonic bonding, adhesive bonding, or the like. The spacer 128 is preferably glued or otherwise attached to the base structure 138 between the backsheet layer 30 and the topsheet layer 31 as representatively illustrated in FIG. 19A. FIG. 19A is a cross-sectional view of the rear portion 23 taken at line 19A-19A of FIG. 19. FIG. 19A shows a base structure 138 formed by a backsheet layer 30 and a topsheet layer 31. The spacer 128, attached to the base structure 138, helps open the entrance 91 and form the volume 93. In this embodiment, the three-dimensional capture structure 80 is made of a three-dimensional composite 212 disposed in the rear portion 23 and in various embodiments may include capture elastics 66. The spacer 128 preferably is glued or otherwise attached to the base structure 138 between the backsheet layer 30 and the topsheet layer 31 as representatively illustrated in FIG. 19A.

In the alternative, the spacer 128 can be otherwise attached as by the glue 147 to the base structure 138, such as to the topsheet layer 31 (FIG. 18B) or to the backsheet layer 30 (not shown). Independent of the layer or layers to which the spacer 128 is mounted, the mounting location on the layer can be selected with some discretion. For example, the spacer 128 can be mounted to the three-dimensional capture structure 80 adjacent the longitudinal end edge 26 of the absorbent pad 20, to rear portion 23, or where rear portion 23 and three-dimensional capture structure 80 meet such as at longitudinal end edge 26. Thus, the spacer 128 may be located in the vicinity of longitudinal end edge 26.

The spacer 128 preferably has a rod-like shape. In the embodiments of FIGS. 18 and 19, the spacer 128 includes an internal filler. The filler is resilient to allow some compression, curving and bending without allowing enough deformation so the three-dimensional capture structure 80 becomes too small, or collapsed. Thus, compressive deformation of the spacer 128, when acted on by compressive forces encountered in normal use, is limited to amounts of deformation which maintain sufficient volume 93 in the three-dimensional capture structure 80 to receive an effective amount of exudates from the body of the wearer. FIG. 19B shows a cross-section across the thickness of one embodiment of spacer 128. In this embodiment the spacer includes an internal filler 146 of fluff formed into a rod-like shape. A first containment layer 148 of cloth, film or other containment material contains the internal filler 146 of fluff compressed therein. A second outer and cylindrical layer 150 of fluff is formed around the first containment layer 148. The second layer 150 of fluff is then enclosed within a second containment layer 152 of cloth, film, or other containment material which forms the outside of the spacer 128. With respect to spacer 128, “fluff” can be a regenerated cellulose fluff such as rayon. The spacer 128 can also be a regenerated cellulose fluff and polyester which have been blended, or any material which is deformable, but sufficiently resilient to function as a spacer. For example, scraps of material from the cutting machines in an absorbent article assembly process may be useable as fluff.

Other techniques of adding a spacer to the three-dimensional capture structure 80 of the present invention are taught in U.S. Pat. No. 6,315,764 to Faulks et al., issued Nov. 13, 2001, the content of which is incorporated herein by reference in its entirety.

The absorbent pad 20 also includes a means for holding the absorbent pad 20 in position within a wearer's undergarment during use. As illustrated in FIGS. 2 and 5, a garment attachment means such as a garment attachment adhesive 60 is disposed on the backsheet layer 30 remote from the absorbent assembly 32. The garment attachment adhesive 60 is desirably located transversely inward of the proximal edges 48 of the side panel members 42 and 44. A peel strip 62 of release paper may be releasably bonded to the garment attachment adhesive 60 prior to use. Other garment attachment means such as mechanical fasteners, for example hook-and-loop fasteners, body attachment adhesive, wings, or the like, or combinations thereof may alternatively be used to hold the absorbent pad 20 in position relative to the undergarment or the wearer.

While the invention has been described in detail with respect to specific embodiments thereof, it will be appreciated that those skilled in the art, upon attaining an understanding of the foregoing, may readily conceive of alterations to, variations of and equivalents to these embodiments. Accordingly, the scope of the present invention should be assessed as that of the appended claims and any equivalents thereto.

1. An absorbent pad configured for attachment in an undergarment, the pad having a rear portion, front portion, garment-facing surface, and body-facing surface, the pad comprising:
a backsheet layer;
a liquid permeable topsheet layer secured to and in facing relation with the backsheet layer;
an absorbent assembly disposed between the backsheet layer and the topsheet layer;
a three-dimensional capture structure disposed in the rear portion of the pad on the body facing surface and adapted to conform to a wearer's gluteal groove when the rear portion of the absorbent pad is positioned in an undergarment adjacent a wearer's perianal region.

2. The absorbent pad of claim 1 further comprising elasticized side retention members disposed outboard of the absorbent assembly.

3. The absorbent pad of claim 2 wherein the elasticized side retention members apply tension to the three-dimensional capture structure and facilitate opening the three-dimensional capture structure.

4. The absorbent pad of claim 2 wherein the three-dimensional capture structure further comprises a spacer.

5. The absorbent pad of claim 2 wherein the three-dimensional capture structure is a unitary structure comprising the backsheet layer and the topsheet layer.

6. The absorbent pad of claim 5 wherein the three-dimensional capture structure further comprises one or more capture elastics.

7. The absorbent pad of claim 5 wherein the liquid permeable topsheet layer is disposed primarily on the body-facing surface; and the backsheet layer is disposed partially on the body-facing surface and partially on the garment-facing surface and the portion of the backsheet layer disposed on the garment-facing surface has an area A1 and the portion of the backsheet layer disposed on the body-facing surface has an area A2; and the ratio A1/A2 is less than about 7 to 1.

8. The absorbent pad of claim 7, wherein the ratio A1/A2 is less than about 6 to 1.

9. The absorbent pad of claim 7, wherein the ratio A1/A2 is less than about 5 to 1.

10. The absorbent pad of claim 7, wherein the ratio A1/A2 is less than about 4 to 1.

11. The absorbent pad of claim 2 wherein the three-dimensional capture structure is a non-unitary structure comprising one or more additional materials attached to the absorbent pad.

12. The absorbent pad of claim 2 wherein the three-dimensional capture structure has a width and a height and the ratio of the width to the height is less than about 4 to 1.

13. The absorbent pad of claim 12, wherein the ratio of the width to the height is less than about 3 to 1.

14. The absorbent pad of claim 12, wherein the ratio of the width to the height is less than about 2 to 1.

15. The absorbent pad of claim 12, wherein the ratio of the width to the height is less than about 1 to 1.

16. An absorbent pad configured for attachment in an undergarment, the pad having a rear portion, front portion, garment-facing surface, and body-facing surface, the pad comprising:
a backsheet layer;
a liquid permeable topsheet layer secured to and in facing relation with the backsheet layer;
an absorbent assembly disposed between the backsheet layer and the topsheet layer;
a garment attachment mechanism disposed on the backsheet layer remote from the absorbent assembly;
elasticized side retention members disposed outboard of the absorbent assembly;
a three-dimensional capture structure disposed in the rear portion of the pad and on the body-facing surface, the three-dimensional capture structure comprising the backsheet layer and the topsheet layer folded and joined to form a generally pyramidal shape and adapted to conform to a wearer's gluteal groove when the rear portion of the absorbent pad is positioned in an undergarment adjacent a wearer's perianal region.

17. The absorbent pad of claim 16 wherein the three-dimensional capture structure defines a volume and the three-dimensional capture structure further comprises one or more capture elastics adapted to at least partially maintain the volume when the rear portion of the absorbent pad is positioned in a wearer's perianal region.

18. The absorbent pad of claim 17 wherein the three-dimensional capture structure further comprises a spacer.

19. The absorbent pad of claim 16 wherein the backsheet layer is present in at least a portion of the side retention members.

20. A method of providing a system of reducing leakage from the rear portion of an absorbent pad comprising:
providing at least one absorbent pad according to claim 1;
providing instructions directing a wearer to attach the absorbent pad in an undergarment and position the absorbent pad such that the three-dimensional capture structure is adjacent the wearer's perianal region;
enabling the wearer to position the absorbent pad in the undergarment by following the instructions so as to reduce rear leakage.
